(19) United States
(12) Patent Application Publication
(10) Pub. No.: US 2011/0108041 A1
(43) Pub. Date: May 12, 2011

(54) NASAL DEVICES HAVING A SAFE FAILURE MODE AND REMOTELY ACTIVATABLE

(76) Inventors: Elliot Sather, San Francisco, CA (US); Arthur Ferdinand, San Jose, CA (US); Michael P. Nevares, Martinez, CA (US); Danny Yu-Youh Lai, San Jose, CA (US); Shapour Golzar, Dublin, CA (US)

(21) Appl. No.: 12/941,734

(22) Filed: Nov. 8, 2010

Related U.S. Application Data
(60) Provisional application No. 61/258,865, filed on Nov. 6, 2009.

(51) Int. Cl. A61F 5/56; A61M 16/00

(52) U.S. Cl. 128/848; 128/207.18

(57) ABSTRACT

Described herein are devices, methods and systems that regulate the failure of a nasal device by including a pre-determined failure mode, thereby minimizing the risk. Also described herein are nasal respiratory devices that may be remotely activated or inactivated to turn on and off an increased resistance to exhalation compared to inhalation.

### CROSS REFERENCE TO RELATED APPLICATIONS

This patent application claims priority to U.S. Provisional Patent Application Aerial No. 61/258,865, filed on Nov. 6, 2009, titled “NASAL DEVICES HAVING A SAFE FAILURE MODE.” This application is herein incorporated by reference in its entirety.

This patent application may be related to the following patents and patent applications listed below. In particular, this patent application may be related to U.S. patent application Ser. No. 12/369,691, filed Feb. 11, 2009, which is a continuation of U.S. patent application Ser. No. 11/811,339, filed Jun. 7, 2007, now U.S. Pat. No. 7,506,649 and U.S. patent application Ser. No. 12/044,868, filed Mar. 7, 2008. In addition, this patent application may be related to U.S. patent application Ser. Nos. 11/805,496 filed on May 22, 2007 and titled “NASAL RESPIRATORY DEVICES;” 11/759,916 filed on Jun. 7, 2007 and titled “LAYERED NASAL DEVICES;” 11/298,339 filed on Dec. 8, 2005 and titled “RESPIRATORY DEVICES;” 11/298,362 filed on Dec. 8, 2005 and titled “METHODS OF TREATING RESPIRATORY DISORDERS;” 11/298,640 filed on Dec. 8, 2005 and titled “NASAL RESPIRATORY DEVICES;” 12/141,875 filed on Jun. 18, 2008 and titled “ADHESIVE NASAL RESPIRATORY DEVICES;” 11/811,401 filed on Jun. 7, 2007 and titled “NASAL RESPIRATORY DEVICES FOR POSITIVE END-EXPIRATORY PRESSURE;” 09/881,862 filed on Jun. 14, 2001 and titled “METHODS AND DEVICES FOR IMPROVING BREATHING IN PATIENTS WITH PULMONARY DISEASE,” (now U.S. Pat. No. 6,722,360); 10/827,073 filed on Apr. 19, 2004 and titled “METHODS AND DEVICES FOR IMPROVING BREATHING IN PATIENTS WITH PULMONARY DISEASE,” (now U.S. 7,334,581); 12/014,060 filed on Jan. 14, 2008 and titled “METHODS AND DEVICES FOR IMPROVING BREATHING IN PATIENTS WITH PULMONARY DISEASE;” 11/941,915 filed on Nov. 16, 2007 and titled “ADJUSTABLE NASAL DEVICES;” 11/941,913 filed on Nov. 16, 2007 and titled “NASAL DEVICE APPLICATORS;” 11/811,339 filed on Jun. 7, 2007 and titled “NASAL DEVICES,” (now U.S. Pat. No. 7,506,649); 12/044,868 filed on Mar. 7, 2008 and titled “RESPIRATORY SENSOR ADAPTERS FOR NASAL DEVICES;” 12/369,681 filed on Feb. 11, 2009 and titled “NASAL DEVICES;” 12/364,264 filed on Feb. 2, 2009 and titled “CPAP INTERFACE AND BACKUP DEVICES;” 12/329,271 filed on Dec. 5, 2008 and titled “PACKAGING AND DISPENSING NASAL DEVICES;” 12/329,895 filed on Dec. 8, 2008 and titled “DELAYED RESISTANCE NASAL DEVICES AND METHODS OF USE;” 12/405,837 filed on Mar. 17, 2009 and titled “NASAL DEVICES WITH NOISE-REDUCTION AND METHODS OF USE;” 12/485,750 filed on Jun. 16, 2009 and titled “ADJUSTABLE RESISTANCE NASAL DEVICES.” All of these patents and patent applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices, systems and methods described herein are related to nasal devices, and particularly the use of nasal devices to treat disorders including sleeping disorders.

### BACKGROUND

Detection and treatment of patients suffering from breathing disorders often requires that the patent's breathing be monitored. Monitoring may be particularly important during treatment, because it allows a physician to estimate the efficacy of treatment, and may permit dynamic modification of the treatment. For example, it may be helpful to monitor respiration in patients suffering from, or at risk for, medical conditions such as snoring, sleep apnea (obstructive, central and mixed), Cheyne Stokes breathing, UARS, COPD, hypertension, asthma, GERD, heart failure, and other respiratory and sleep conditions. Sleep labs may monitor patients to diagnose these and other conditions of sleep disordered breathing. Monitoring typically involves taping a sensor to the subject or applying a mask including a sensor over the subject's nose and/or mouth.

Unfortunately, applying a sensor to a subject in this fashion may be uncomfortable, and may make it even harder for the patient to sleep, confounding the diagnosis and treatment. This may be particularly true when sensors are used in combination with treatments involving a medical device that is worn on the subject's face, nose, and/or mouth. If a separate sensor is used, it may be difficult to match the sensor to the treatment system, which may add to patient discomfort, as the monitoring device and the treatment device must both be worn concurrently. In addition to the loss of comfort, combining sensing and treatment systems may also result in a loss of accuracy, as sensing may interfere with the function of treatment systems. Such problems may persist even with currently available treatment systems that include an integrated monitoring sensor or sensors.

Recently, devices and methods for treating breathing disorders using a passive airflow resistor have been developed. These devices are typically much smaller and lighter and therefore may be more comfortable. Examples of these devices may be found in U.S. patent application Ser. Nos. 11/298,640, titled “NASAL RESPIRATORY DEVICES” (filed Dec. 8, 2005); U.S. patent application Ser. No. 11/298,339, titled “RESPIRATORY DEVICES” (filed Dec. 8, 2005); U.S. patent application Ser. No. 11/298,362, titled “METHODS OF TREATING RESPIRATORY DISORDERS” (filed Dec. 8, 2005); U.S. patent application Ser. No. 11/805,496, titled “NASAL RESPIRATORY DEVICES” (filed May 22, 2007); U.S. patent application Ser. No. 11/811,339, titled “NASAL DEVICES” (filed Jun. 7, 2007); U.S. patent application Ser. No. 11/759,916, titled “LAYERED NASAL DEVICES” (filed Jun. 7, 2007); U.S. patent application Ser. No. 11/811,401, titled “NASAL RESPIRATORY DEVICES FOR POSITIVE END-EXPIRATORY PRESSURE” (filed Jun. 7, 2007); U.S. patent application Ser. No. 11/941,915, titled “ADJUSTABLE NASAL DEVICES” (filed Nov. 16, 2007); and U.S. patent application Ser. No. 11/941,913, titled “NASAL DEVICE APPLICATORS” (filed Nov. 16, 2007). Each of these references was previously incorporated by reference in its entirety.

FIGS. 1A and 1B illustrate one variation of a nasal respiratory device having a passive airflow resistor. In FIGS. 1A and 1B, the nasal respiratory device includes an airflow resistor 105 that is positioned in a central passageway through the device. The airflow resistor in this example is a flap valve device. The airflow resistor is configured so that the expiratory airflow through the passageway has a higher resistance than inspiratory airflow. For example, the flap valve 109 opens virtually completely during inspiration to allow airflow through the device, but remains closed during expiration (as shown in FIG. 1A). The flap valve is prevented from opening during expiration by two (or more) flap valve limiters 111 which at least partially span the passageway. The nasal device of FIGS. 1A and 1B also includes two leak pathways 107, 107′, which remain open even during expiration. Careful configuration of the leak pathways and airflow resistors allows the resistance and/or flow rates during inspiration and expiration to be controlled. For example, a nasal respiratory device may include a resistance to expiration that is between about 0.01 and about 0.25 cm H₂O/ml/sec and a resistance to inhalation that is between about 0.0001 and about 0.05 cm H₂O/ml/sec when the resistance is measured at 100 ml/sec.

A nasal respiratory device typically also includes a holdfast that secures the device to the nose, so that the airflow resistor is in communication with the nasal passageway. In FIGS. 1A and 1B the holdfast is an adhesive holdfast 101 that extends from the central passageway and allows the flexible attachment of the device to the nose. Other types of holdfasts, including compressible or compliant holdfasts that at least partially insert into the nose, may also be used.

The passageway of the nasal device shown in FIGS. 1A and 1B is a stiff body region that is formed from an inner body rim 117 (in FIG. 1B) and an outer body rim 115 (in FIG. 1A). Other nasal respiratory devices may not include a stiff (or semi-stiff or flexible) rim. The inner body region 117 may also act as an aligner that helps position the device in the nose.

Nasal respiratory devices such as the nasal device shown in FIGS. 1A and 1B may be used to treat a number of respiratory disorders, including sleep disordered breathing such sleep apnea and/or snoring. However, because these devices are worn over the subject's nose, monitoring breathing while wearing the device may be difficult.

In general, nasal devices (e.g., adhesive nasal devices) may be used in conjunction with one or more additional devices, including respiratory monitors or connectors for respiratory monitors. However, if such devices are connected to a nasal device, it may increase the stress on the device, and could potentially lead to failure of the device, or separation of the device into component parts. This is of particular concern when the device includes two or more connected regions that lock together. For example, the device may be a two-part device as illustrated in FIGS. 1A-1B. Failure of the device may be dangerous, particularly if the device, while being worn in the nose, were to break into pieces small enough to be inhaled.

In addition, it would be useful to be able to remotely activate and inactivate the nasal devices, which may be particularly useful in determining and enhancing patient compliance. For example, it may be beneficial to activate (turn “on”) the resistance to exhalation after a patient has begun sleeping. Remote activation of such devices may be particularly useful.

Described herein are devices, methods and systems that either prevent such failure of a nasal device, or regulate the failure by including a pre-determined failure mode, thereby minimizing the risk. Also described herein are nasal respiratory devices that may be remotely activated or inactivated to turn on and off an increased resistance to exhalation compared to inhalation.

### SUMMARY OF THE INVENTION

The present invention relates to nasal devices having a predetermined failure mode. In particular, described herein are nasal devices for use with one or more additional structures that may be attached to the nasal device, including connectors, sensors, adaptors, or the like. In general, these devices may be configured so that application of stress and/or strain (e.g., by attachment to of an additional device such as a cannula, adapter, or the like and/or force applied by a user either consciously or unconsciously during operation of the device) will result in a predictable failure of the device. The failure mode may prevent the device from fragmenting or breaking apart in a manner that could result in harm or injury to the patient. In particular, the nasal devices described herein may be configured to have a failure mode in which the connection between the holdfast and the airflow resistor component (including any housing or valve body for the airflow resistor) fail by separating.

Thus, described herein are nasal devices having a body formed of two or more body components (e.g., rim body regions) and a holdfast region that are configured so that force applied to the device will preferentially result in the separation of the holdfast region from the body region. In some variations, the body region will be configured so that the components of the body region (e.g., rim body) are secured together with greater strength than the connection between the body region and the holdfast region. For example, the holdfast region may be an adhesive holdfast that is secured to or between two halves of a rim body region. Pulling on one portion of the rim body will result in separating the entire rim body (all components) from the holdfast region.

Also described herein are systems, devices and methods for remotely turning a nasal respiratory device on or off. For example, described herein are systems for remotely activating/inactivating a nasal device to turn on or off the increase in resistance to exhalation compared to the resistance to inhalation. Remote activation typically means that the nasal device may be turned on or off by someone other than the patient wearing the device. For example, a system for remotely turning a nasal device on or off may include: a nasal device configured to be sealed in communication with a subject's nasal orifice without covering the subject's mouth, the nasal device having a passive airflow resistor configured to inhibit exhalation more than inhalation through the nasal device; and a remotely activatable control configured to remotely activates an interfering member to prevent the airflow resistor from substantially increasing resistance through the nasal device during inhalation, and that remotely inactivates the interfering member so that it does not prevent the airflow resistor from increasing the resistance through the nasal device during inhalation.

In general the nasal devices described herein may be referred to as “passive” nasal devices or nasal devices having passive airflow resistors. The devices typically inhibit exhalation through the nasal device using an airflow resistor that does not apply positive (or negative) pressure through the application of additional respiratory gas. Instead, these devices may use a valve mechanism to inhibit inhalation more than exhalation.

Thus, in some variations of the system, the nasal device comprises an adhesive nasal respiratory device. The passive airflow resistor may comprise a flap valve.

In some variations, the remotely activatable control is a pneumatically activatable control, or an electrically activatable control, or the like.

In some variations, the interfering member comprises a mechanical occluder. For example, the interfering member may comprise a Nitinol loop.

The systems described herein may also include support configured to support the interfering member adjacent to the nasal respiratory device. For example, a support may be an over-mask support configured to support the interfering member adjacent to the nasal respiratory device. A support may be an adapter configured to connect to the nasal device and to support the interfering member adjacent to the nasal respiratory device.

Also described herein are remote control devices for remotely activating and inactivating a nasal device having a passive airflow resistor that increases the resistance to exhalation through the nasal device more than the resistance to inhalation through the nasal device, the remote control device comprising: an interfering member configured to prevent the airflow resistor from substantially increasing resistance through the nasal device during inhalation; a support member for supporting the interfering member adjacent to the nasal device so that the interfering member may engage the airflow resistor when the interfering member is activated; and a remotely activatable control configured to remotely activate the interfering member.

As mentioned, the interfering member may comprise a mechanical occluder, such as a Nitinol loop.

The support may comprise an over-mask support configured to be worn over the nasal device to support the interfering member adjacent to the nasal respiratory device. In some variations, the support comprises an adapter configured to connect to the nasal device and to support the interfering member adjacent to the nasal respiratory device.

The remotely activatable control may be a pneumatically activatable control. The remotely activatable control may be an electrically activatable control.

Also described herein are methods comprising remotely activating a nasal device having a passive airflow resistor that is configure to inhibit exhalation through the device more than inhalation through the device.

### BRIEF DESCRIPTION OF THE DRAWINGS

FIGS. 1A and 1B show bottom (external) and top (internal) perspective views of a nasal respiratory device

FIGS. 2A and 2B show distal views of two variations of a nasal respiratory device adjacent to a nasal cannula opening.

FIGS. 3A and 3B show perspective views of a sensor adapter connected to a nasal respiratory device.

FIGS. 4A and 4B show bottom and top perspective views, respectively, of a sensor adapter.

FIGS. 4C and 4D show top and bottom views, respectively, of the sensor adapter shown in FIGS. 4A and 4B.

FIG. 5 shows a pair of nasal device to which a cannula has been attached (using a sensor adapter such as the one shown in FIGS. 4A-4D).

FIG. 6A-6C shows a nasal device having a multiple-component valve body (a 2-part valve body in this example), in which the upper and lower valve body are adapted to secure together so that the adhesive holdfast region will separate from the combined valve body before the valve body will separate into component parts. FIG. 6A shows an exploded view of the upper (“inner”) portion of a valve body above the lower (“outer”) portion of the valve body. A flat circumferential adhesive holdfast extends from the valve body. FIG. 6B shows the nasal device of FIG. 6A fully assembled. FIG. 6C is a partial cross-section through the nasal device of FIGS. 6A-6B.

FIG. 7 illustrates components of one variation of a system for remote activation of a nasal respiratory device. In this variation, the system describes a nasal device with an on/off mechanism comprising a remotely activatable Nitinol loop that is hydraulically activated.

FIG. 8 shows the system of FIG. 7 being worn by a subject. In this variation, the subject is wearing two nasal respiratory devices (one one adhesive secured to each nostril) as well as the hydraulically activated remote on/off mechanism that is supported by the modified over-mask worn over the nose.

FIGS. 9A and 9B show an adhesive nasal device having an airflow resistor that may be remotely activated/inactivated by a hydraulically controlled loop of material (Nitinol) that can extend into the airflow resistor to either interfere by holding the airflow resistor open during exhalation (FIG. 9B) or withdrawn interference and allow the airflow resistor to inhibit exhalation more than inhalation (FIG. 9A).

FIGS. 10A-10D illustrate another system for remotely activating/inactivating (on/off) a nasal respiratory device as described herein. In this variation a nasal cannula adapter similar to that shown in FIGS. 4A to 4D is configured to include remotely activatable element (e.g., Nitinol loop) that maybe controlled (e.g., pneumatically) to turn on/off the expiratory resistance through the nasal device. This variation, in contrast to the example sown in FIGS. 7-8B, may be directly attached to each adhesive nasal device without the need for an over-mask. FIGS. 10A and 10B show side perspective views of a remotely activatable on/off controller for a nasal respiratory device illustrating both the “off” (FIG. 10A) and “on” (FIG. 10B) states. Similarly, FIGS. 10C and 10D show side views of the devices shown in FIGS. 10A and 10B, respectively.

FIG. 11A is one example of a remote controller for a remotely activatable on/off controller for a nasal respiratory device, as described herein. In FIG. 11A, the system includes a pneumatically activatable controller for extending/retracting the interfering member (Nitinol wire or loop) into the airflow resistor of one or more nasal respiratory devices. FIG. 11B shows the master controller (master pneumatic cylinder) for this variation.

FIG. 12A shows the slave cylinder portion of the pneumatic remote controller system of FIG. 11A. FIGS. 12B and 12C illustrate the slave cylinder in the off and on configurations, respectively.

### DETAILED DESCRIPTION OF THE INVENTION

Described herein are nasal devices having one or more predetermined failure modes. For example, described herein are nasal devices in which the connections between the components regions of the device are calibrated so that force applied to the nasal device will result in breaking the nasal device in a predetermined fashion. In some variations, the nasal devices are configured so that force applied to a region of the nasal device (e.g., one portion of a rim body of the device), will result in separation of the holdfast (e.g., adhesive holdfast) from the rest of the nasal device (e.g., valve body). This may be achieved by connecting the various components of the nasal device, but particularly the holdfast region and the multi-component valve body, so that the weakest connection is between the holdfast and the rest of the valve body, or between the valve body and other portions of the valve body, or between the holdfast and another portion (e.g., a break-away portion) of the holdfast or valve body. Thus, in some variations, the device includes a break-away region configured to fail before other region of the device when force is applied to the device. In any of the variations described herein, the phrase “when force is applied” may refer to the application of force to cause failure of the device. This application of force may be is applied via the connection of another structure to the nasal device. The other structure may be a sensor, sensor adapter, cannula, etc.

For example, a nasal device may be used with a sensor adapter that attaches to the nasal device in a way that could provide additional undesired force to the nasal device. For example, if the sensor adapter is a cannula attachment device (cannula adapter), when the cannula is attached, tension along the length of the cannula may apply force to the nasal device and particularly to the rim body of the nasal device when the cannula adapter is configured to attach to just one portion of the rim body (e.g., the outer rim body). Thus, as the patient wearing the device and cannula moves around during sleep (or awake), the cannula may pull/push, or otherwise apply force to nasal device at the attachment site (e.g., the lower rim body).

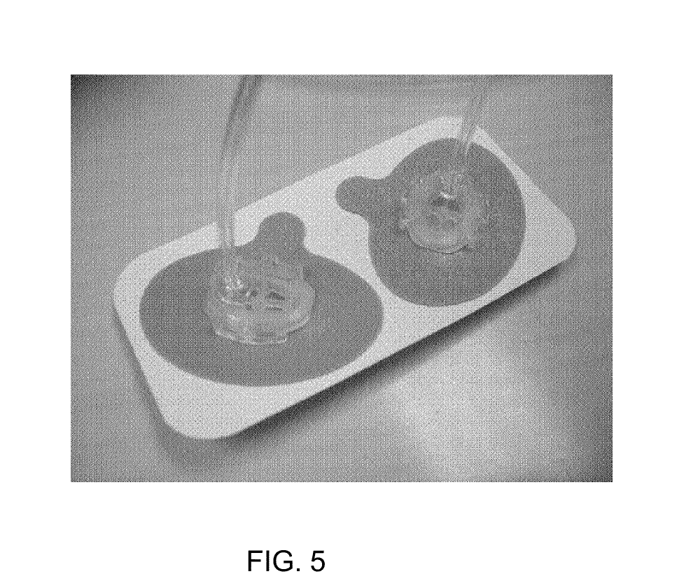

An example of this is illustrated in FIG. 5, showing a nasal device with attached cannula.

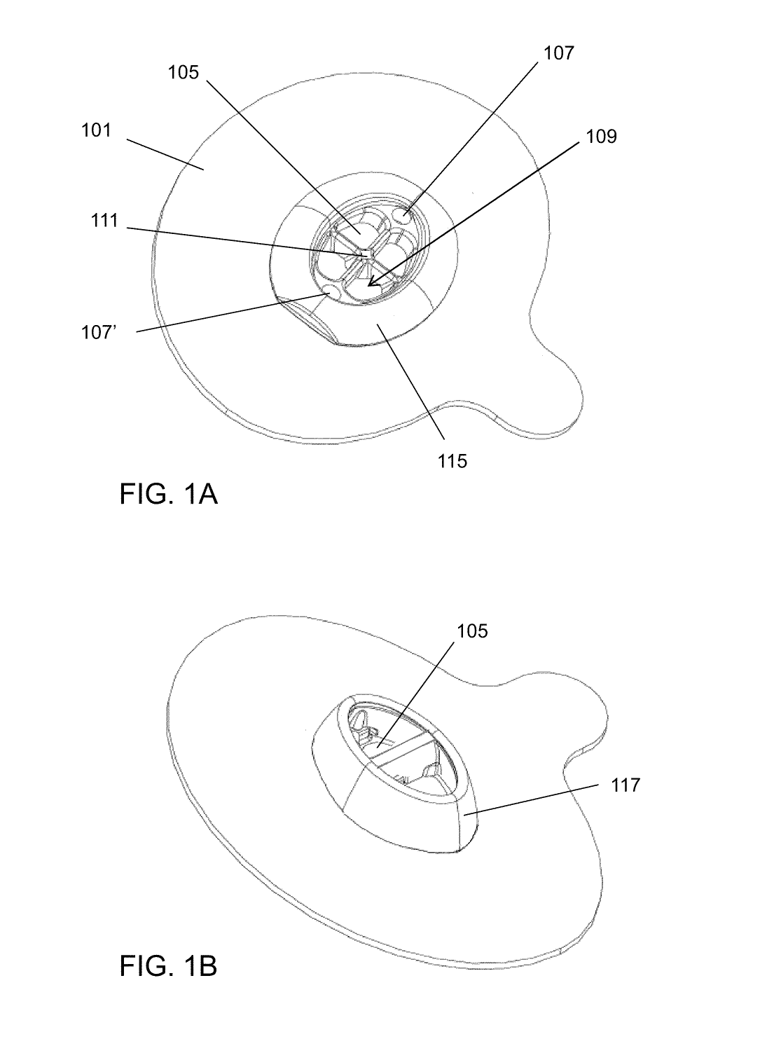

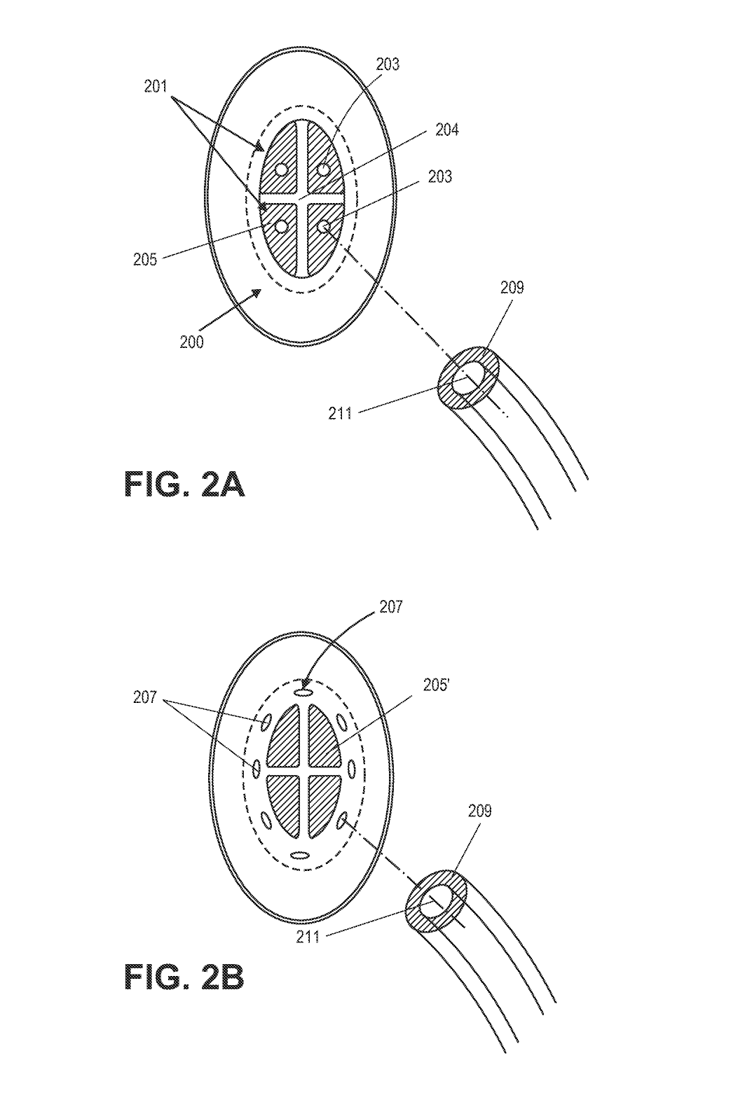

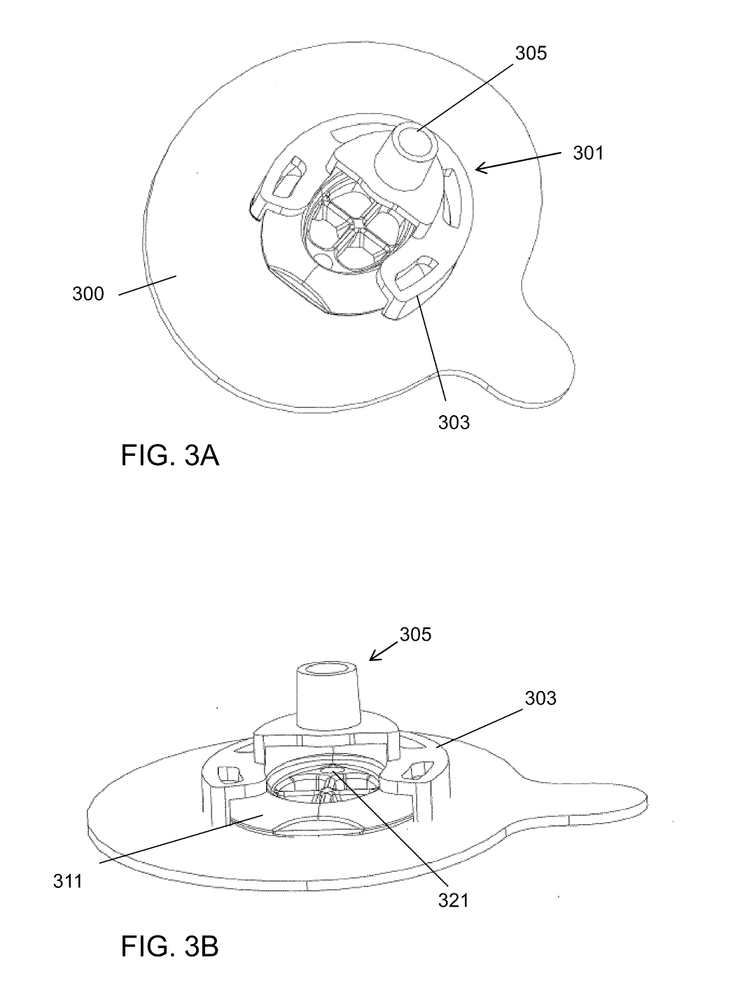

In general, the sensor adapters described herein may be used with one or more nasal respiratory devices, particularly nasal respiratory devices that include a passive airflow resistor. An example of a nasal respiratory device is shown in FIGS. 1A and 2A, described above. Other examples may also be found in the following U.S. patent applications, each of which was previously incorporated by reference in its entirety: U.S. Ser. No. 11/298,640, titled “Nasal Respiratory Devices”; U.S. Ser. No. 11/805,496, titled “Nasal Respiratory Devices”; U.S. Ser. No. 11/811,339, titled “Nasal Devices”; U.S. Ser. No. 11/759,916, titled “Layered Nasal Devices”; U.S. Ser. No. 11/811,401, titled “Nasal Respiratory Devices for Positive End-Expiratory Pressure”; and U.S. Ser. No. 11/941,915, titled “Adjustable Nasal Devices.”

FIGS. 2A and 2B illustrate how respiration could be monitored when using a nasal respiratory device 200. Generally, a sensor 209 may be held near or against the distal face of the nasal respiratory device to measure respiration through the nasal device. The sensor detector input of the sensor should be held secured in approximately the same region, and it should not substantially alter the function of the nasal respiratory device. Finally, the distance from an opening (or openings) through the nasal respiratory device to the sensor may be important to detecting accurate readings. A sensor adapter (not shown in FIGS. 2A and 2B) may be used to reliably secure a sensor relative to the nasal device without substantially altering the function (or resistance to expiration and inspiration) of the nasal device.

In order to get reproducible sensor readings when using a passive-resistance nasal respiratory device, it may be helpful to place the sensor in communication with more than one outlet of a nasal device. In particular, the sensor detector input may be placed in communication with an expiratory outlet (e.g., leak pathway) and a valved outlet. A valved outlet is the opening through the nasal device that is typically regulated by the airflow resistor so that it is closed (or partially closed) during expiration. Placement of the nasal device in communication with just an expiratory outlet may result in an imbalance in the magnitude of the sensor reading between inspiration and expiration, since the airflow during inspiration is typically distributed between both leak pathways and the valved openings (which are typically much larger) and during expiration the airflow is predominantly limited to the leak pathways. By positioning the sensor detector input in communication with both a leak pathway (or expiratory outlet) and valved pathway openings, the signals during both expiration and inspiration may be more balanced. In some variations the proximity of the sensor detector input to either a leak pathway and/or a valved pathway opening is determined by the ratio of the sizes of the opening; the sensor detector input may be closer to the smaller of the two openings, typically the leak pathway/expiratory outlet.

Similarly, the distance from the opening(s) and the sensor detector input of the sensor may be predetermined. If the sensor detector input is too close to an opening of the nasal respiratory device it may interfere with operation of the nasal respiratory device; if it is too far, it may not accurately sense respiration. Thus, in some variations the sensor detector input is greater than 1 mm from the nasal device outlet (e.g., leak pathway opening and/or valved opening), or greater than 2 mm away, or between 1 mm and 10 mm away.

It should be understood that when the specification refers to positioning a sensor with respect to the nasal device (e.g., in communication with an outlet of the nasal device), the region of the sensor positioned is the sensor detector input, unless the context makes clear otherwise.

It is desirable to measure respiration through the nasal device during both inspiration and expiration. A sensor can be placed in communication with one or more openings of the nasal respiratory device to measure one or more characteristic of respiration through the nasal device. As described in greater detail below, any appropriate sensor may be used, including a pressure sensor connected to a cannula, a thermister, a thermocouple, etc. A cannula 209 (connected to a pressure sensor, not shown) having an opening 211 is illustrated in FIGS. 2A and 2B.

As mentioned, the position of the sensor detector input (e.g., cannula 209) relative to the openings in the nasal device on the external side may dramatically affect the accuracy and stability of the sensor readings. For example, it may be useful to measure airflow from an expiratory opening in the nasal respiratory device. In FIGS. 2A and 2B the openings in the nasal respiratory devices are leak pathways 203, 207. In FIG. 2A the leak pathway is formed thorough the flap valve 205. In FIG. 2B the eight leak pathways are formed separately from the flap valve. In either case, this expiratory opening allows airflow during exhalation when the airflow resistor is at least partially closed.

The body frame of the sensor adapter may control the distance between a sensor (including cannula) and the external side of the nasal respiratory device. Further, the body frame of the sensor adapter is typically configured so that is does not interfere with the operation of the nasal respiratory device to which attaches. This means that the sensor adapter does not substantially limit flow through the passive nasal respiratory device to which it attaches. For example, a passive nasal respiratory device typically increases the resistance to expiration greater than the resistance to expiration, and may maintain these resistances within a predetermined range.

Returning to the exemplary passive nasal respiratory device shown in FIGS. 1A and 1B, the nasal respiratory device includes an airflow resistor 105. In this example, the airflow resistor is a flap valve 105, although any appropriate airflow resistor (e.g., ball valve, etc.) may be used. When worn by a subject, the airflow resistor increases the resistance to expiratory airflow by closing at least partially during expiration. Thus, during expiration, airflow through the device passes predominantly (or completely) through the leak pathways 107, 107′. During inspiration the airflow resistor 105 is open, and inspiratory airflow may pass through the valved opening 109 in addition to the leak pathways 107, 107′. The valved opening in this example is divided into four parts by the support struts/flap valve limiter 111. FIG. 1A shows the distal, or external side of the nasal respiratory device. When worn by a subject, the external side of the nasal device faces outward, and airflow into and out of the nasal respiratory device passes through the leak pathways 107, 107′ and the valved opening 109.

A sensor adapter typically attaches to the external side of a nasal respiratory device, such as the external side of the devices shown in FIGS. 1A, 2A and 2B. The sensor adapter body frame is configured so that it attaches on the external side of the airflow resistor so that a sensor can be secured in communication with at least a portion of an opening on the nasal respiratory device. The opening is generally an inspiratory and/or expiratory opening, such as a leak pathway 107, 107′ in FIGS. 1A and 203 and 207 in FIGS. 2A and 2B, respectively, or a valved opening 109.

The body frame of the sensor adapter is also configured so that it can attach to the nasal respiratory device without substantially altering the function (e.g., the inspiratory or expiratory resistance) of the nasal respiratory device. For example, the body frame of the sensor adapter may project only slightly over an opening of the nasal respiratory device when the sensor adapter is attached to the nasal respiratory device. Alternatively, or in addition, the body frame may include one or more openings (e.g., windows, gaps, passages, etc.) to allow airflow from the opening(s) of the nasal respiratory device to communicate with the outside environment substantially unimpeded.

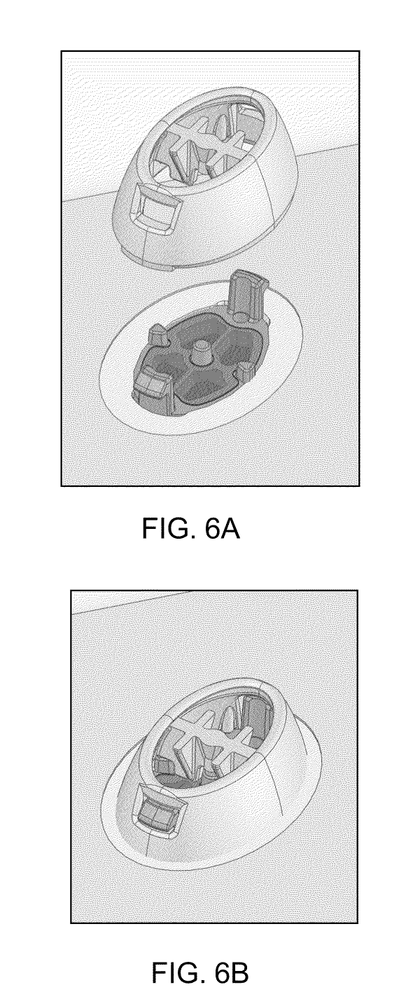

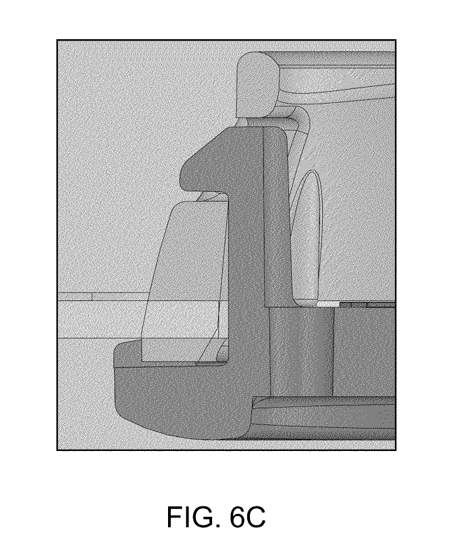

FIGS. 6A through 6C illustrate one variation of a nasal device adapted to have a predetermined failure mode. In this example, the snap connecting the upper and lower rim body regions is stronger than the connection between the rim body and the adhesive. Thus, force applied to the nasal device by a connected adjunct device (such as a sensor adapter, cannula, sensor, or the like) will result in failure of the connection with the adhesive before separation of the rim body in to component parts. In some variations the material or structure of holdfast itself (not necessarily the connection between the holdfast and the valve body or other regions) is configured to fail. For example, the holdfast may be configured to tear or rip before the valve body separates or before the connection to the valve body and the holdfast fails, or before the connection between other regions of the device (e.g., the upper and lower valve bodies), even when the force is applied at the lower valve body, or at some other portion of the device. In some variations the force is applied at the junction between an adapter (e.g., for an auxiliary device) and the rest of the nasal device.

As mentioned above, the device of FIGS. 6A-6B is modified to include a relatively robust connector (e.g., snap) between the upper and lower valve body. For example, the length of the connector (snap) arm is relatively long (extending almost the entire thickness of the device) and the material forming the valve body is relatively stiff/rigid. For example, the body (and particularly the arms forming the robust connector/snap) are formed of polycarbonate in this example, which may be stiffer than a material such as polypropylene, for example, while retaining the ease of molding.

### Remotely Controlled On/Off Regulator

Also described herein are remotely controlled on/off regulators for nasal devices, including any of the nasal respiratory devices described herein. In particular, described herein are systems and devices for remotely activating (turning “on”) and inactivating (turning “off”) a nasal respiratory device having an airflow resistor.

In general, these systems may be referred to as remotely controlled on/off regulators for nasal respiratory devices. A typical remotely controlled on/off regulator system may include an occluding member (e.g., mechanical occluder) that interferes with the airflow resistor to hold or block the airflow resistor open, in an inactivated state. In the normal course of operation the nasal device described herein include an airflow resistor that is open during inhalation (providing low resistance, if any, to inhalation) and closed, constricted or partially closed during exhalation (providing an increased resistance to exhalation compared to inhalation). For example, a nasal respiratory device may include an airflow resistor having a valve such as a flap valve (or flap valves), a ball valve, or the like. A remotely controlled on/off regulator for a nasal respiratory device may therefore include a mechanical occlude that may be remotely controlled to interfere or be withdrawn from interference with the airflow resistor of one or more (e.g., two) nasal respiratory devices.

For example, in some variations the remotely controlled on/off regulator for a nasal respiratory device includes a mechanical occluder that is a remotely extendable/retractable Nitinol member that holds the airflow resistor(s) of the nasal device either open (inactive or “off”) or is withdrawn from interference (active of “on”). In some variations this occluder is a loop or loops of Nitinol.

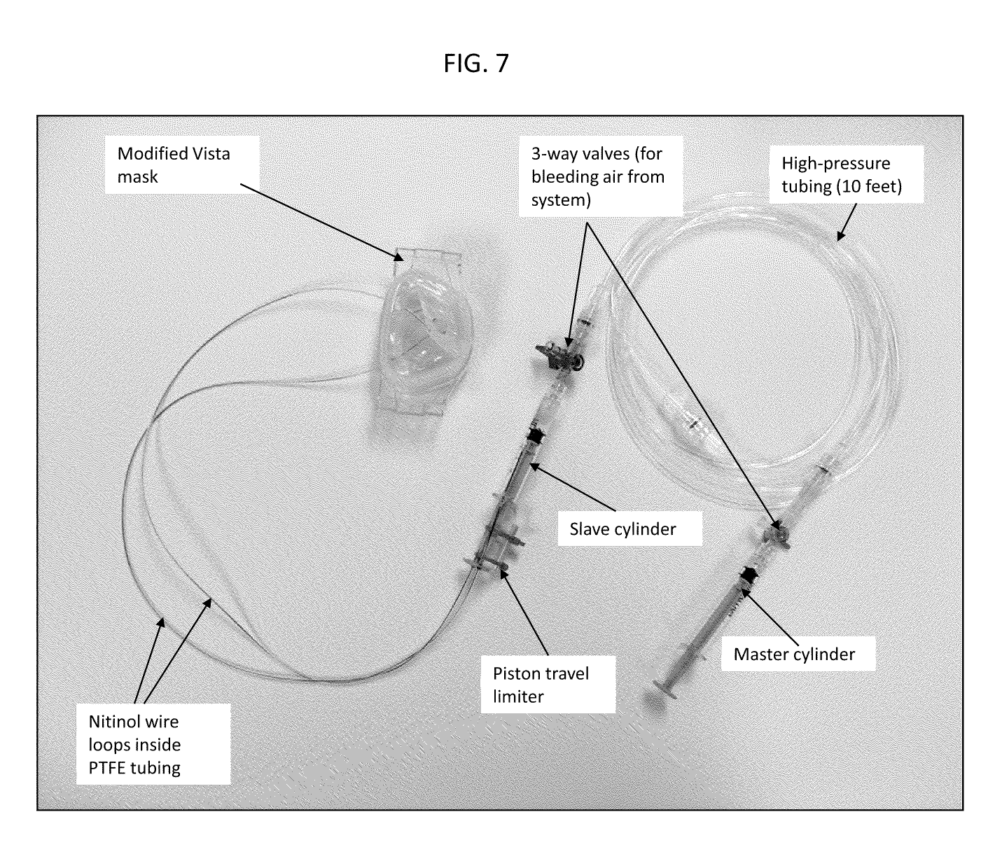

FIG. 7 shows one variation of a remotely controllable on/off regulator for a nasal respiratory device, configured as a hydraulic actuator that activates/inactivates the nasal device by moving a mechanical occluder (comprising a loop of Nitinol) into or out of interference with an airflow resistor. The system may be controlled using a master remote controller that may be positioned some distance from the patient wearing the nasal device (e.g., greater than 2 feet, greater than 5 feet, greater than 10 feet, etc.).

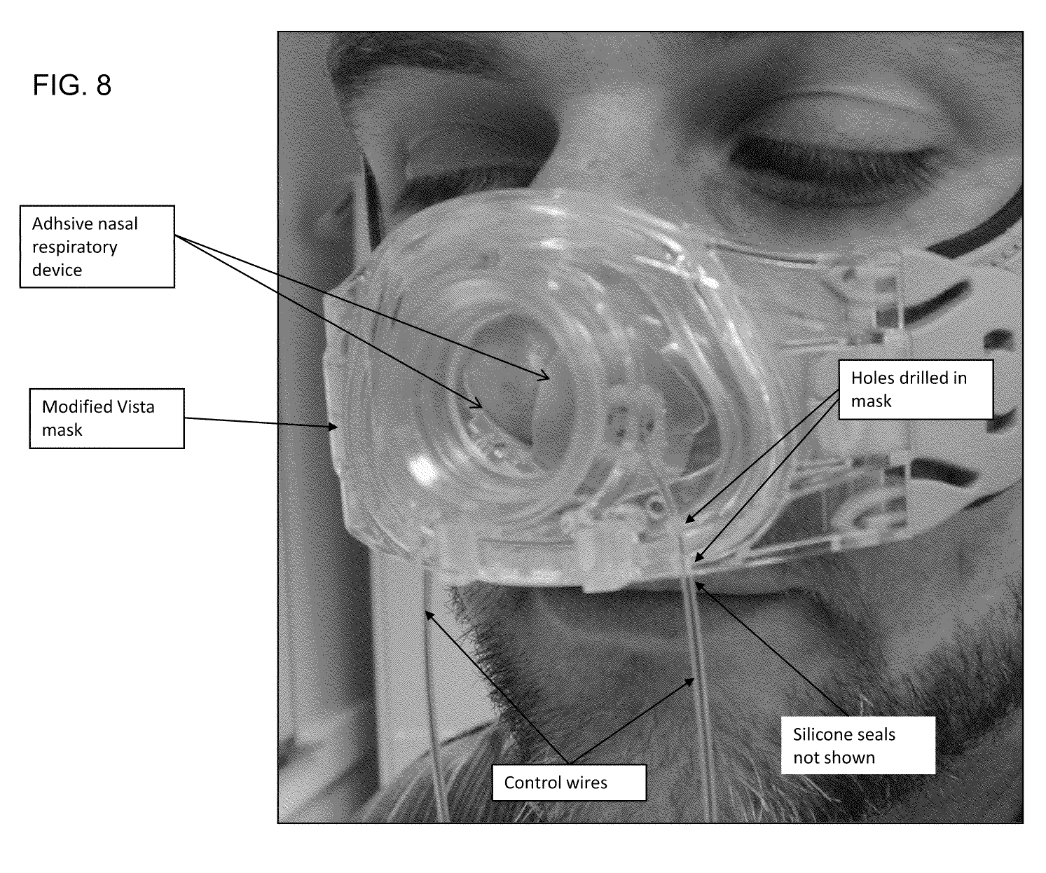

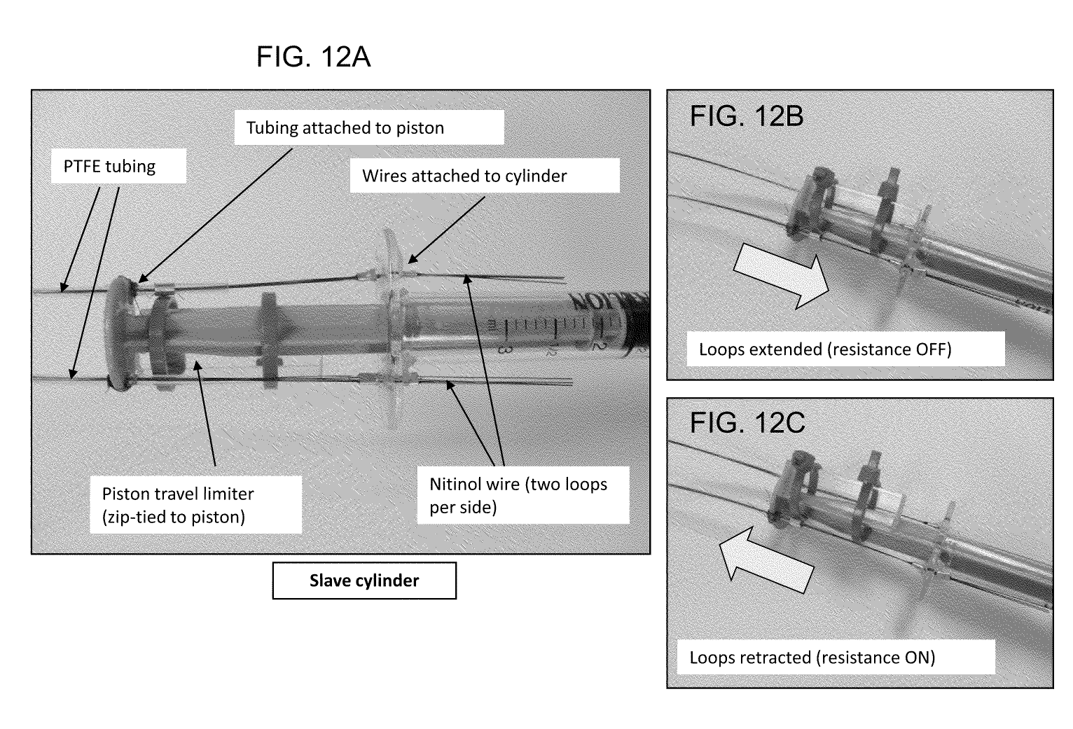

In FIG. 7, the system includes an occluder comprising a loop of Nitinol wire that is supported adjacent to the nasal device to be regulated by a support. In this variation the support is an over-mask formed by modifying a nasal mask (a “Vista” mask) which fits over the patient's nose when the patient is also wearing one or more adhesive nasal respiratory devices, as illustrated in FIG. 8. The system also includes a hydraulic control for activating/inactivating the occluder, causing the occluder to move into the nasal airflow resistor to interfere and prevent it from closing and increasing resistance to exhalation more than inhalation, or to cause the occluder to move out of the nasal airflow resistor and not prevent the airflow resistor from increasing the resistance to exhalation. The hydraulic control comprises Nitinol wire loop(s) inside PTFE tubing that is connected to a piston travel limiter for extending/retracting the wire. The piston traveler is coupled to a slave cylinder that may be pneumatically controlled over a long length of tubing (e.g., 10 feet) and connected to a master cylinder for remote control. Additional variations and illustrations of this system may be seen in FIGS. 11A-12C.

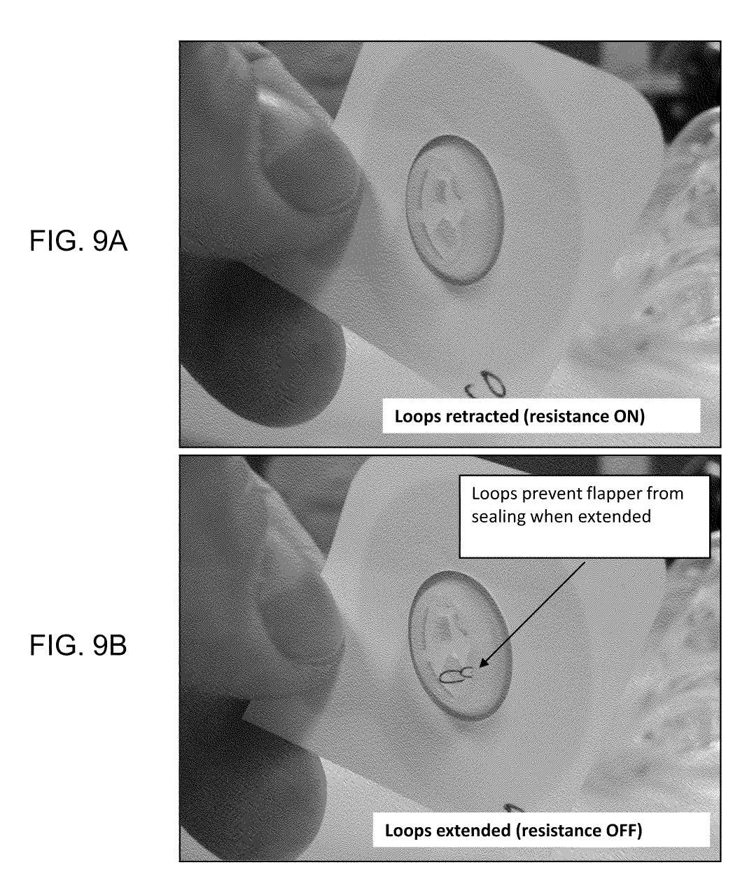

FIGS. 9A and 9B illustrate the activation of the mechanical occluder shown in FIG. 7. For example, in FIG. 9A the mechanical occluder (a loop of Nitinol) is retracted, so that the device is “on” and (when worn by a subject), the airflow resistor may provide an enhanced resistance to exhalation compared to inhalation. In FIG. 9B the Nitinol wire loop is extended to occlude the airflow resistor, preventing the airflow resistor for inhibiting exhalation more than inhalation. In this variation, the airflow resistor comprises a flap valve that is prevented from sealing when the loop of Nitinol is extended by the remotely activated controller.

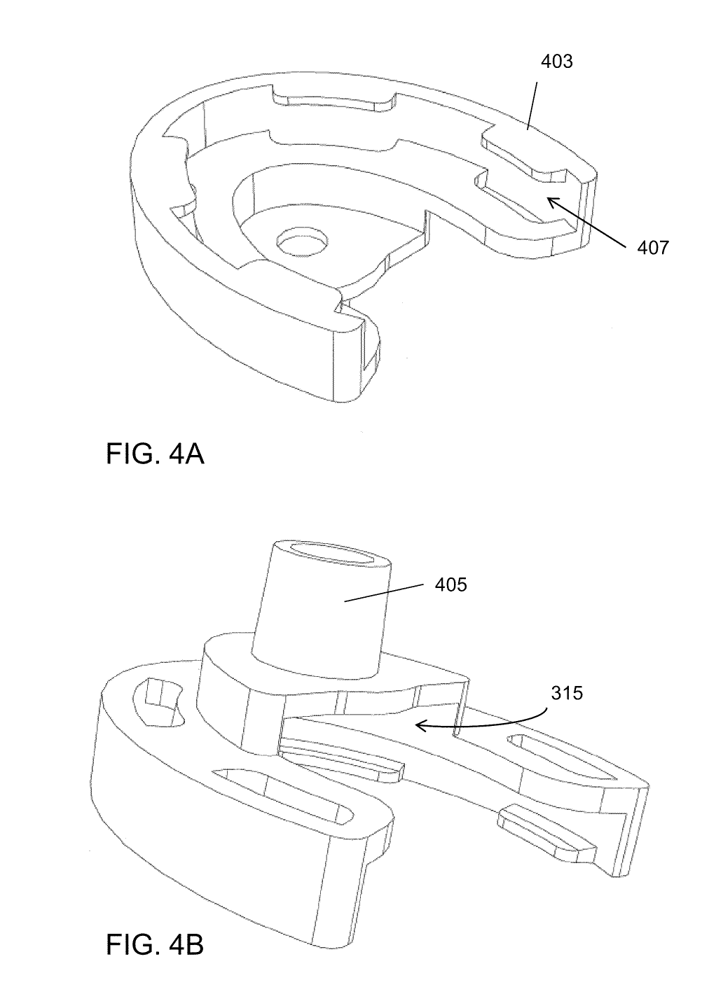

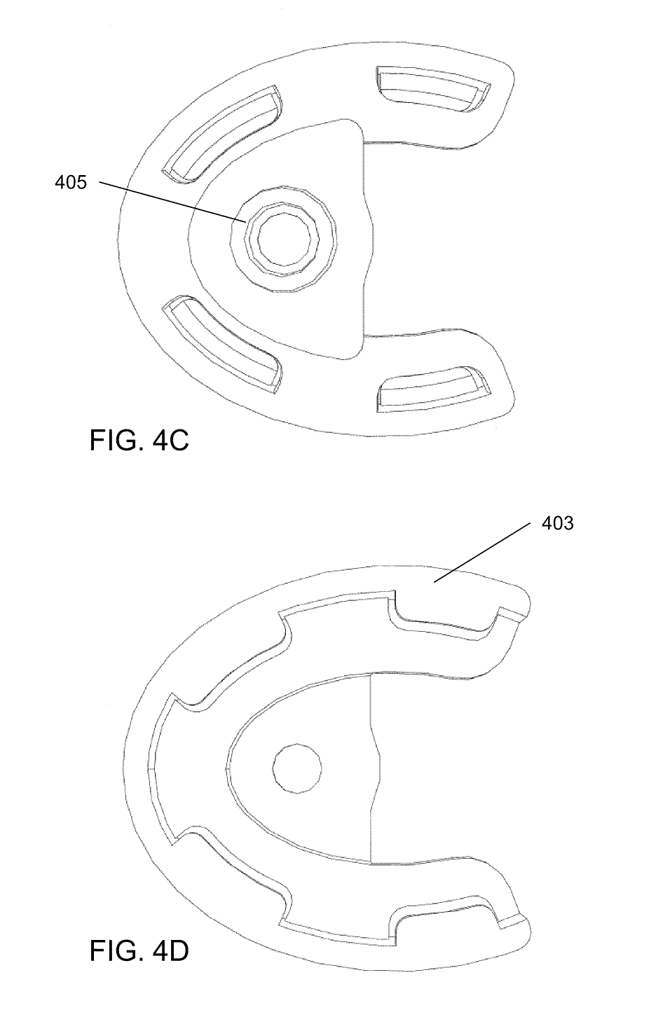

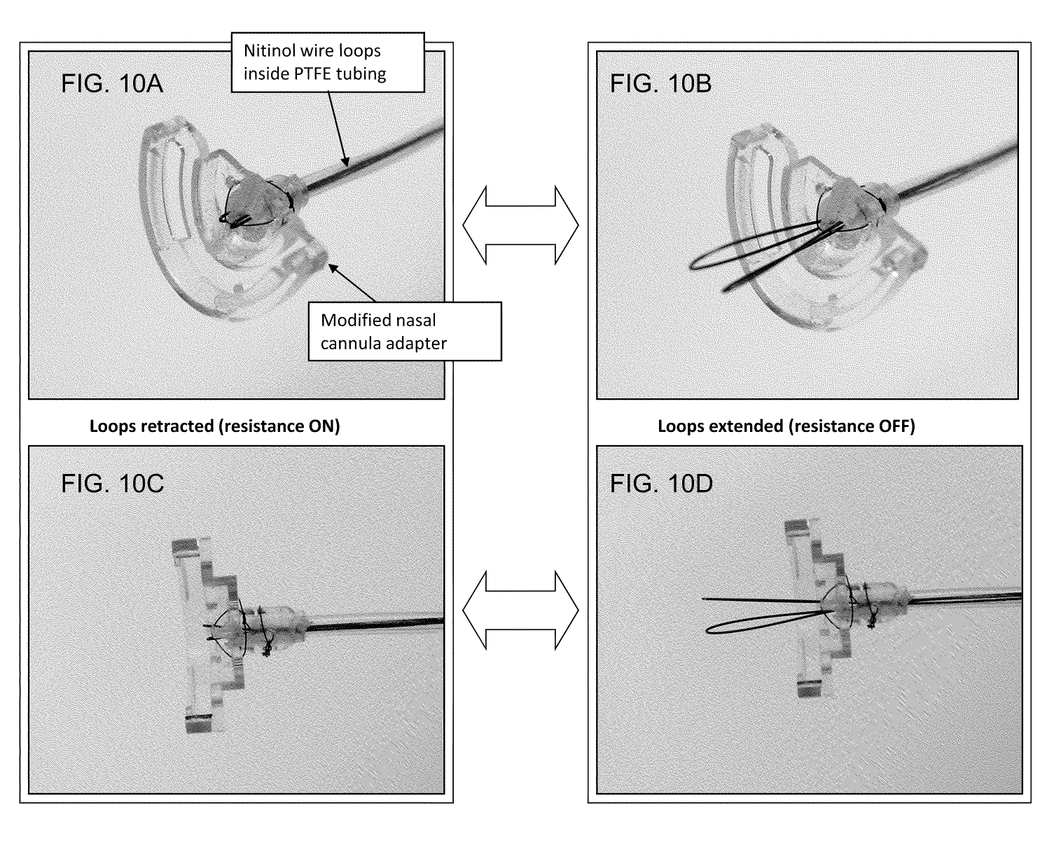

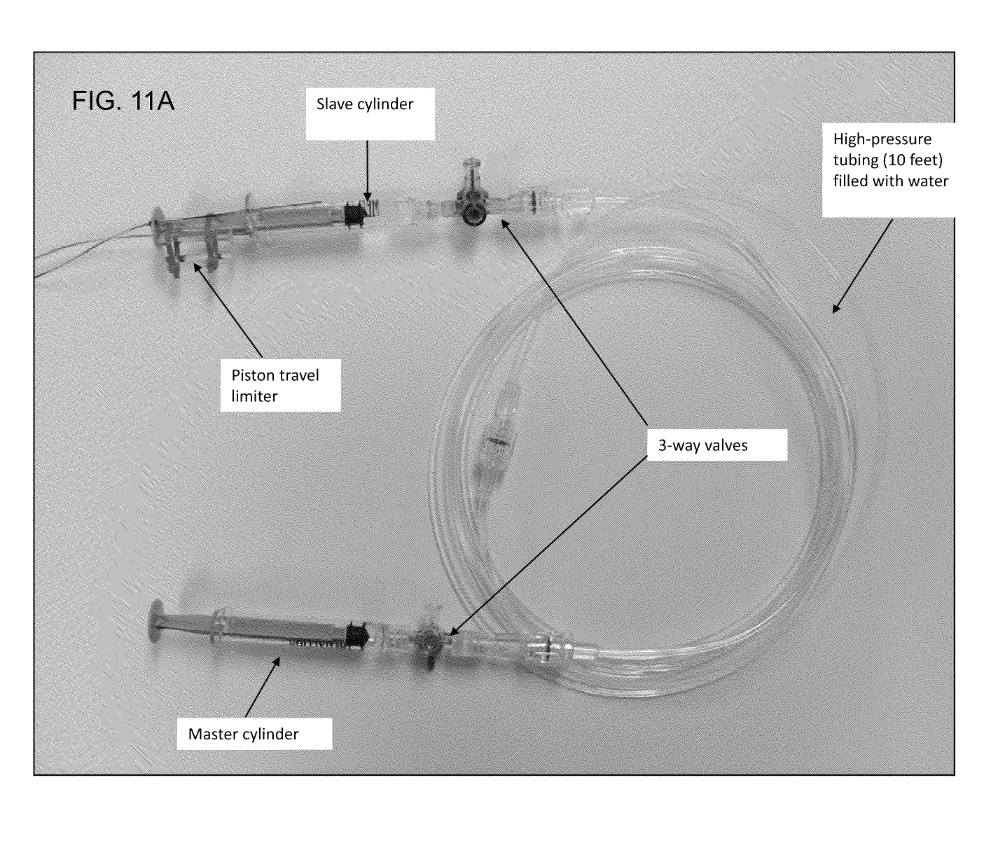

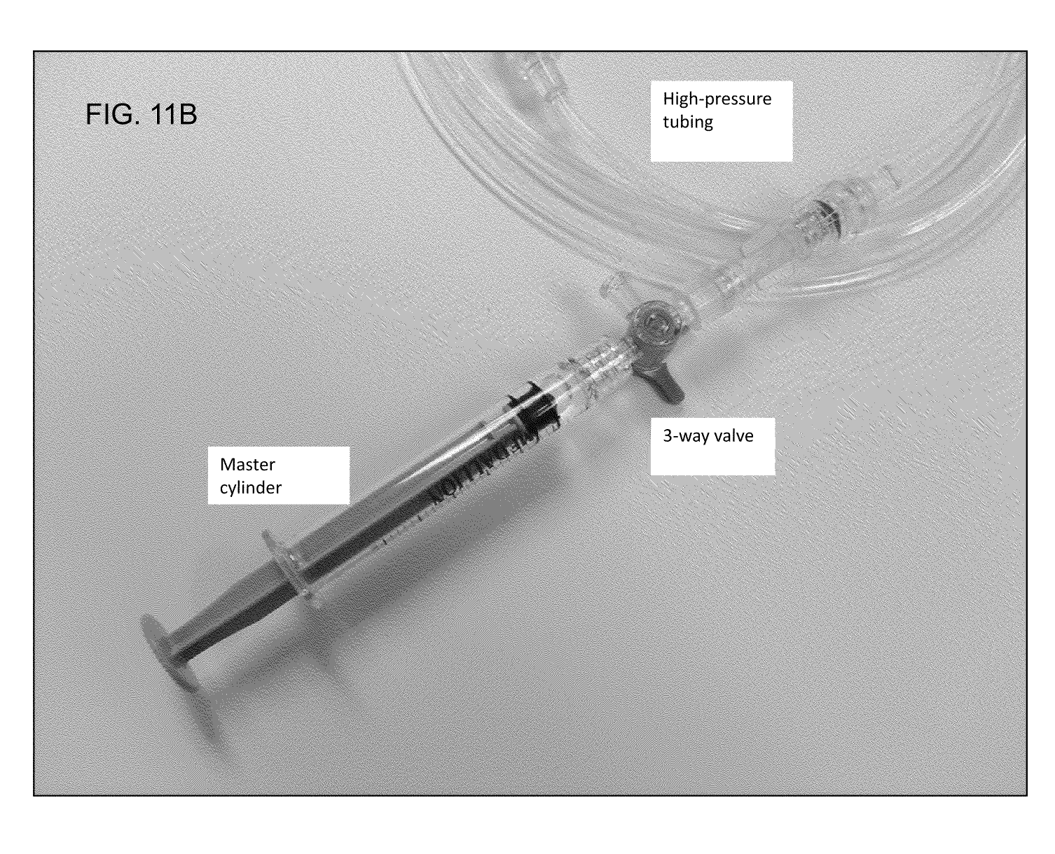

Another variation of a pneumatically activated remotely controlled on/off regulator for a nasal respiratory device is illustrated in FIGS. 10A-10D. In this variation the mechanical occluder is supported adjacent to the nasal device to be regulated by a support comprising an adapter similar to the nasal cannula adapter illustrated and described above in FIGS. 4A-4D. The other components of the system, including the mechanical occluder, the pneumatic control and the master remote controller may be similar or identical to those illustrated above in FIGS. 7 and 11A-12C. In FIGS. 10A-10D, the loop of Nitinol comprising the mechanical occluder is shown in the “off” position in FIGS. 10A and 10C, and in the “on” (extended and interfering) position in FIGS. 10B and 10D.

In some variations the occluder is a mechanical occlude that is electrically remotely controlled. For example, an occluder may be extended by a solenoid or other member than can be switched from an “on” or “off” position by wireless or wired electrical means. For example, a nasal cannula adapter such as that shown in FIGS. 4A-4D may be adapted to include a wireless receiver and a loop of material that may be extended or retracted by activating a solenoid. When the wireless receiver receives a signal to turn “on” the solenoid may withdraw the mechanical occluder (e.g., Nitinol loop, post, etc.) from interference with the airflow resistor. If the wireless receiver receives a signal to turn “off” the solenoid may extend the mechanical occluder to interfere with the airflow resistor. An on-board power supply (e.g., battery) may also be provided.

In operation, a nasal respiratory device may be remotely controlled (turned on/off) using any of the systems described herein to remotely activate or inactivate the airflow resistor. “Remote activation” typically means control of activation/inactivation by a user other than the person wearing the device (e.g., the patient). The remote activation may occur while the subject is sleeping. For example, a subject may wear one or more nasal respiratory devices configured to inhibit exhalation more than inhalation. Prior to falling asleep the nasal respiratory device may be remotely controlled to inactivate the device, turning it off, so that exhalation is not inhibited substantially more than exhalation by the airflow resistor. After the patient has fallen asleep a third party (e.g., doctor, researcher, technician, sleeping partner) may remotely turn the device “on.” This may remotely cause the withdrawal of an interfering member from the airflow resistor, allowing the airflow resistor to operate to increase the resistance to exhalation more than inhalation.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

We claim:

1. A system for remotely activating/inactivating a nasal device to turn on or off the increase in resistance to exhalation compared to the resistance to inhalation, the system comprising:
a nasal device configured to be sealed in communication with a subject's nasal orifice without covering the subject's mouth, the nasal device having a passive airflow resistor configured to inhibit exhalation more than inhalation through the nasal device; and
a remotely activatable control configured to remotely activates an interfering member to prevent the airflow resistor from substantially increasing resistance through the nasal device during inhalation, and that remotely inactivates the interfering member so that it does not prevent the airflow resistor from increasing the resistance through the nasal device during inhalation.

2. The system of claim 1, wherein the nasal device comprises an adhesive nasal respiratory device.

3. The system of claim 1, wherein the passive airflow resistor comprises a flap valve.

4. The system of claim 1, wherein the remotely activatable control is a pneumatically activatable control.

5. The system of claim 1, wherein the remotely activatable control is an electrically activatable control.

6. The system of claim 1, wherein the interfering member comprises a mechanical occluder.

7. The system of claim 1, wherein the interfering member comprises a Nitinol loop.

8. The system of claim 1 further comprising a support configured to support the interfering member adjacent to the nasal respiratory device.

9. The system of claim 1, further comprising an over-mask support configured to support the interfering member adjacent to the nasal respiratory device.

10. The system of claim 1, further comprising an adapter configured to connect to the nasal device and to support the interfering member adjacent to the nasal respiratory device.

11. A remote control device for remotely activating and inactivating a nasal device having a passive airflow resistor that increases the resistance to exhalation through the nasal device more than the resistance to inhalation through the nasal device, the remote control device comprising:
an interfering member configured to prevent the airflow resistor from substantially increasing resistance through the nasal device during inhalation;
a support member for supporting the interfering member adjacent to the nasal device so that the interfering member may engage the airflow resistor when the interfering member is activated; and
a remotely activatable control configured to remotely activate the interfering member.

12. The device of claim 11, wherein the interfering member comprises a mechanical occluder.

13. The device of claim 11, wherein the interfering member comprises a Nitinol loop.

14. The device of claim 11, wherein the support comprises an over-mask support configured to be worn over the nasal device to support the interfering member adjacent to the nasal respiratory device.

15. The device of claim 11, wherein the support comprises an adapter configured to connect to the nasal device and to support the interfering member adjacent to the nasal respiratory device.

16. The device of claim 11, wherein the remotely activatable control is a pneumatically activatable control.

17. The device of claim 11, wherein the remotely activatable control is an electrically activatable control.

18. A method comprising: remotely activating a nasal device having a passive airflow resistor that is configure to inhibit exhalation through the device more than inhalation through the device.
